(19) United States
(12) Patent Application Publication
(10) Pub. No.: US 2011/0262567 A1
(43) Pub. Date: Oct. 27, 2011

(54) COMPOSITION FOR TREATING GOUT, CONTAINING ANGELICA GIGAS EXTRACT HAVING A XANTHINE OXIDASE-INHIBITING EFFECT AND AN INFLAMMATION-INDUCING ENZYME-INHIBITING EFFECT

(75) Inventors: Jae Seon Kang, Busan (KR); Jin Young Lee, Busan (KR); Yun Jung Park, Busan (KR); Jae Yeon Park, Busan (KR); Kang Min Kim, Busan (KR); Min Hui Park, Busan (KR); Ik Hwan Kim, Seoul (KR); Yong Geun Hong, Gyeongsangnam-do (KR); Min Su Jeong, Gyung Sang Nam Do (KR); Woo Sik Jeong, Busan (KR); Seon Ok, Busan (KR)

(73) Assignee: KOREA BIO HEALTH CO., LTD., GYEONGSANGNAM (KR)

(21) Appl. No.: 13/141,631

(22) PCT Filed: Dec. 16, 2009
(86) PCT No.: PCT/KR09/07549

§ 371 Date: Jun. 22, 2011

(30) Foreign Application Priority Data
Dec. 23, 2008 (KR) 10-2008-0132592

(51) Int. Cl. A61K 36/232; A61P 19/06

(52) U.S. Cl. 424/725

(57) ABSTRACT

The present invention relates to a composition for the treatment of gout, comprising an Angelica gigas Nakai extract which inhibits xanthine oxidase to reduce uric acid in the blood or urine, thereby being effective in the treatment of gout.

Further, the present invention relates to a composition for the prevention of inflammation in gout, comprising an Angelica gigas Nakai extract which has an inhibitory effect on the inflammation-inducing enzyme Cox-2.

The Angelica gigas Nakai extract of the present invention is a liquid concentrate consisting of 98% by weight or more of decursin and decursinol angelate as main ingredients, and for administration to human, it is extracted with water and ethanol only as an extraction solvent. In addition, a purification method using a difference in temperature and solubility is used, in order to increase its purity.

### TECHNICAL FIELD

The present invention relates to a composition for the treatment of gout, comprising an Angelica gigas Nakai extract which inhibits xanthine oxidase to reduce uric acid in the blood or urine, thereby being effective in the treatment of gout.

Further, the present invention relates to a composition for the prevention of inflammation in gout, comprising an Angelica gigas Nakai extract which has an inhibitory effect on the inflammation-inducing enzyme Cox-2.

### BACKGROUND ART

Angelica gigas Nakai is native to Korea, and has been used as a blood-nourishing agent in oriental medicine. Recent studies have reported that the indicators for Korean angelica, decursin and decursinol invigorate blood circulation and have an anti-helicobacter activity, and the present inventors demonstrated that purified decursin shows the efficacies in inhibition of renal toxicity, prevention of renal failure due to diabetes, and diabetic hypertension in the animal experiment (PCT/KR1999/00632).

Further, the present inventors registered an extraction method of Angelica gigas Nakai in Korean Patent No. 10-0509843. They improved this, and then also applied the patent regarding an extraction method of Angelica gigas Nakai to include 95% by weight or more of a main ingredient and its antioxidant effects (Korean patent application No. 10-2007-45441).

Moreover, the present inventors applied the patent regarding the extracts of Angelica gigas Nakai and Angelica gigas Jiri having an improving effect on lipid metabolism and an extraction method thereof (Korean Patent application No. 10-2008-4122).

The present inventors also developed a concentration method capable of obtaining a large amount of extract in a simple manner, compared to the known method, and prepared a liquid formation using this method. They demonstrated that oral administration of this liquid formation to laboratory animals is effective for intestinal absorption.

Up to date, decursin and its structural isomer decursinol angelate have not been known well, but the present inventors have studied their actions, and established an analysis method thereof (Korean Patent No. 10-0509843).

The present inventors revealed that decursin may exist as a solid at room temperature, but decursinol angelate exists as a liquid even at −70° C., and the ratio of decursin and decursinol angelate in Angelica gigas Nakai is about 57:43. They also analyzed its accurate content and ratio by a mass analysis, and the results revealed that it is a purified material consisting of 50˜54.5% of decursin, 40˜42% of decursinol angelate, 0.1˜0.3% of decursinol, about 2˜3% of decursinol derivative, and 1˜2% of other ingredients.

Gout is a metabolic disease in which a high level of uric acid maintains in the blood, and thus uric acid crystals are not removed from the body and accumulate in the tissues to cause various symptoms. An increased uric acid level in the blood may also occur due to excessive production of uric acid and the kidneys' inability to remove the uric acid. The increased uric acid level in the blood does not directly cause gout, but it easily forms and accumulates the uric acid crystals in various tissues, which develops into gout by risk factors for 10˜20 years. Gout commonly occurs in men aged 40 to 50, and the age of onset has been getting younger according to dietary changes and environmental factors, and the risk of gout increases in women after menopause or in women with renal failure because of long term use of diuretic or immunosuppressive agents after transplantation.

The causes of gout have been known as follows. Most cells constituting our body have the nucleus, which consist of nucleic acids including genetic information. The nucleic acids consist of purines or pyrimidines. On cell death, purines in the nucleus are broken down to produce a large amount of uric acid, and sodium urate crystals accumulate in the tissue, which causes inflammation such as hyperuricemia, gouty arthritis, gouty renal disease, gouty renal calculus or the like (Yagi K., Chem. Phys. Lipids, 45, p337, 1987).

During purine metabolism, adenine is converted into uric acid via hypoxanthine and xanthine, and guanine is directly metabolized into xanthine, and then converted into uric acid, in which xanthine oxidase is involved. When xanthine oxidase converts xanthine into uric acid, a large amount of superoxide radical is generated, which imposes oxidative stress to surrounding cells, and thus causes gout, as well as hypertension, hyperlipidemia, arteriosclerosis, diabetes or the like (Storch J et al., Anal. Biochem., 169, p262, 1988).

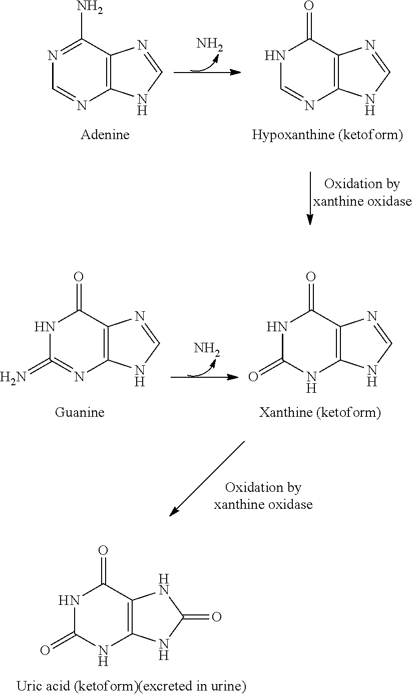

Uric acid synthesis can be largely divided into extrinsic (derived from purines included in ingested foods) and intrinsic (derived from those formed in the body) pathways. Most of the produced uric acid is excreted in the urine by the kidney through a complicated process, and a loss of balance between production and excretion generates a problem. A normal blood level of uric acid is 7˜8 mg/dl or less in men, and 6 mg/dl or less in women.

Even if there is a difference in the blood level of uric acid between each individual, the uric acid blood level over 8 mg/dl causes pain and is injurious to the heart or kidney, or causes cerebrovascular disorders, and the uric acid blood level over 10 mg/dl causes convulsion. The most common symptom of gout is arthritis, but it may develop into acute or chronic renal failure.

Colchicines (when the diagnosis is in question), non-steroidal anti-inflammatory drugs (when the diagnosis is clear without any complications, but it may cause side-effects of gastric ulcer, decreased kidney function, edema or the like), and steroids (intra-articular or intramuscular injection for monoarthritis) are mainly prescribed for the treatment of gout.

Unfortunately, there have been no therapeutic agents capable of successfully treating gout in modern medicine. There have been known allopurinol as an anti-hyperuricemic agent and probenecid as a uricosuric drug, but allopurinol has undesirable side-effects including skin rash, gastroenteric trouble, bone marrow suppression, itching, nausea, muscle pain or the like. Therefore, there is a need to develop a safe substance capable of preventing or treating gout with fewer side-effects.

### DISCLOSURE

### Technical Problem

Since the known therapeutic agents for gout have severe side-effects, it is intended to provide a novel therapeutic composition for the treatment and prevention of gout without causing side-effects.

### Technical Solution

In order to achieve the above object, it is an object of the present invention to provide a composition for the treatment of gout and a composition for the prevention of inflammation in gout, comprising an Angelica gigas Nakai extract that includes decursin and decursinol angelate as an active ingredient, in which decursin and decursinol angelate show an inhibitory activity on xanthine oxidase and an inflammation-inducing enzyme Cox-2 and reduce the uric acid level increased by potassium oxonate.

### Advantageous Effects

The Angelica gigas Nakai extract of the present invention inhibits the activity of xanthine oxidase to reduce the increased level of uric acid in gout, and also inhibits the activity of an inflammation-inducing enzyme Cox-2 in gout.

### DESCRIPTION OF DRAWINGS

FIG. 1 shows an Angelica gigas Nakai extract comprising 98% by weight of the extracted and purified decursin and decursinol angelate.

FIG. 2 shows an Angelica gigas Jiri extract comprising 98% by weight of the extracted and purified decursin and decursinol angelate.

FIG. 3 is a graph showing uric acid levels in the serum and urine of a normal control, a hyperuricemia control group (model control), an allopurinol-treated group (positive control), and an Angelica gigas Nakai extract-treated group (Decursin 50).

FIG. 4 shows inhibitory effects of a normal control, a hyperuricemia control group (model control), an allopurinol-treated group (positive control), and an Angelica gigas Nakai extract-treated group (Decursin 50) on the inflammation-inducing enzyme, Cox-2 (cyclooxygenase-2).

### BEST MODE

The present invention relates to an Angelica gigas Nakai extract which has an inhibitory effect on xanthine oxidase to reduce the uric acid level in the blood or urine, thereby being effective for the treatment of gout. Further, the present invention relates to an Angelica gigas Nakai extract having an inhibitory effect on the inflammation-inducing enzyme.

The Angelica gigas Nakai extract of the present invention was prepared according to the method of Korean Patent Application No. 10-2007-45441 applied by the present inventors.

1. Preparation of Angelica gigas Nakai extract

The raw material, Angelica gigas Nakai native to South Korea and North Korea was pulverized to 40 mesh or less, and dried to a moisture content of 5% or less, followed by extraction for 12 hrs or longer with addition of medicinal alcohol or ethanol in a volume of 2 to 4-fold of the pulverized Angelica gigas Nakai. The extract was filtered and quantified. The content of decursin and decursinol angelate in this concentrate was 33%.

The concentrate was evaporated to dryness, and thus a highly viscous material was obtained. 1 liter of medicinal alcohol or ethanol per 1 kg of the material was added to dissolve the main ingredient, and left at −20° C. for 10 hrs. The produced precipitate was removed by centrifugation to obtain the supernatant. The supernatant was evaporated to dryness, and thus a concentrate was obtained.

The concentrate was eluted with 50 liters of 60% ethanol. The content of decursin and decursinol angelate in this concentrate was 330 grams, and the amount of main ingredient to be dissolved in 50 liters of 60% ethanol was also 330 grams.

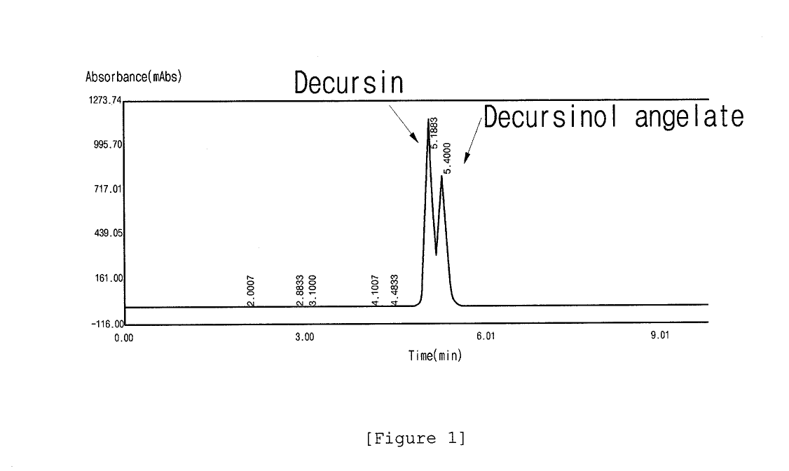

After elution, centrifugation was performed to obtain a 60% ethanol layer. This ethanol layer was evaporated to dryness, so as to obtain a concentrate. This concentrate was dissolved in 99% alcohol, and the supernatant was collected to obtain a final concentrate (see FIG. 1).

The concentrated Angelica gigas Nakai extract was analyzed by liquid chromatography (HPLC), and the results showed that the total content of decursin and decursinol angelate was 98% by weight. The following experiment was performed using this extract.

2. Test on Xanthine Oxidase Inhibition

In this experiment, xanthine was used as a substrate and xanthine oxidase was used as an enzyme to perform an enzyme-substrate reaction. The inhibitory activity on xanthine oxidase was determined according to the method of Stripe F. et al., (J.Biol. Chem, 244., p3855-3863, 1969).

250 μl of 50 mM potassium phosphate buffer (pH 7.5), 385 μl of Angelica gigas Nakai extract solution (prepared by dilution with 50 mM potassium phosphate buffer (pH 7.5) until the final concentrations of decursin and decursinol angelate become 1, 5, 10, 17.5, 20, 50 μg/ml), and 330 μl of xanthine solution were mixed with each other to prepare a reaction solution.

35 μl of xanthine oxidase solution (final concentration of 0.4 U/ml) was added to the reaction solution, and reacted in a 37° C. incubator for 15 min. Then, absorbance (Ab) was measured at 295 nm.

Each reaction solution was also prepared using each Angelica gigas Nakai extract (each final concentration of decursin and decursinol angelate was 1, 5, 10, 17.5, 20, or 50 μg/ml) without addition of the xanthine oxidase solution. Further, in the absence of the Angelica gigas Nakai extract, each reaction solution was prepared with or without the xanthine oxidase solution. During preparation of these reaction solutions, 50 mM potassium phosphate buffer (pH 7.5) was added in a volume corresponding to that of the Angelica gigas Nakai extract or xanthine oxidase, until the volume of final reaction solution was 1 ml.

The xanthine oxidase inhibition ratio of the present invention was calculated by the following Equation, and the results are shown in Table 1.

Inhibition ratio (%)=(1−B/A)×100

A: a change in absorbance in the absence of composition (Absorbance with enzyme−Absorbance without enzyme)

B: a change in absorbance in the presence of composition (Absorbance with enzyme−Absorbance without enzyme)

TABLE 1

| | Xanthine Oxidase | |
|---|---|---|
| Sample | Inhibition ratio (%) | |
| concentration | of Angelica gigas | |
| (μg/ml) | Nakai extract | IC₅₀ (μg/ml) |
| 1 | 6.48 ± 0.13 | 18.2 |
| 5 | 10.69 ± 0.93 | |
| 10 | 35.47 ± 1.43 | |
| 17.5 | 40.39 ± 1.40 | |
| 20 | 76.72 ± 2.98 | |
| 50 | 93.80 ± 1.47 | |

When allopurinol was used as a standard inhibitor at a concentration equal to that of the Angelica gigas Nakai extract, its inhibitory effect was found to be IC₉₀ of 38.0 μg/ml.

That is, the known xanthine oxidase inhibitor allopurinol was found to have IC₅₀ of 38.0 μg/ml, but the Angelica gigas Nakai extract of the present invention was found to have IC₅₀ of 18.2 μg/ml. Further, the known literature reported that allopurinol has IC₉₀ of 80˜100 μg/ml, but the Angelica gigas Nakai extract of the present invention was found to have IC₉₀ of approximately 44.7 μg/ml, suggesting that the composition of the present invention shows a more excellent inhibitory effect on xanthine oxidase.

The above IC₅₀ value means the concentration providing 50% inhibition of xanthine oxidase, and the composition of the present invention was found to show the most excellent inhibitory effect, as compared to 100 μg/ml of green tea extract, Crataegus oxyacantha extract, kelp extract, Zizyphus jujuba extract, and Ginkgo biloba extract showing the xanthine oxidase inhibition ratio of 92%, 85%, 68%, 64%, and 78% (Korean Patent No. 10-0492470), 30 μg/ml of old red platycodon showing the xanthine oxidase inhibition ratio of 63.6% (Korean Patent No. 10-0662206), and Salicornia herbacea extract showing the xanthine oxidase inhibition ratio of IC₅₀ of 21 μM (Korean Patent No. 10-0569244).

3. Test on Uric Acid Reduction in Blood and Urine of Gout-Induced Animal Model

In order to confirm anti-gout activity of the Angelica gigas Nakai extract of the present invention, an uricase inhibitor, potassium oxonate was administered to induce hyperuricemia, and then a reduction in the blood uric acid by the Angelica gigas Nakai extract was analyzed. As a positive control, a gout disease model administered with an inhibitor of uric acid synthesis, allopurinol (Sam Il Pharm.) was used to perform a comparative experiment.

As the experimental animal, 9-week old male sprague-dawley rats (250-280 g, Samtako, Inc.) were acclimated in a SPF chamber where the humidity and temperature were maintained at 50±5% and 24˜26° C. and feed and water were supplied ad libitum for 1 week, and then used for the experiment.

In the present experiment, the uricase inhibitor, potassium oxonate (150 mg/kg) was intraperitoneally administered one day before sample treatment, so as to induce hyperuricemia. The contents of uric acid before and after administration of potassium oxonate were measured and compared to each other, in order to confirm the hyperuricemia induction by administration of potassium oxonate.

Potassium oxonate was suspended in 0.5% sodium carboxymethylcellulose (CMC—Na, 0.5% CMC with 0.1 M sodium acetate (pH5)), and used.

For the experiment, hyperuricemia-induced experimental animals were divided into four groups, and each group consists of 7 rats: a normal control, a hyperuricemia control group (model control), an allopurinol-treated group (positive control), and an Angelica gigas Nakai extract-treated group (Decursin 50).

The hyperuricemia control group was administered with an equal amount of solvent, instead of the Angelica gigas Nakai extract. Each of allopurinol and Angelica gigas Nakai extract was suspended in 0.01 M phosphate buffered saline containing 0.1% polyoxyethylene sorbitane monoleate, and orally administered for 3 days after hyperuricemia induction.

The allopurinol-treated group was administered at a commercial dose of 50 mg/kg, and the Angelica gigas Nakai extract was also orally administered at a dose of 50 mg/kg for 3 days. Within 2 hrs after the last oral administration, the urine was collected from each experimental animal, and immediately autopsy was performed to separate the serum. In order to determine the uric acid level, the uric acid levels in the serum and urine were measured (test equipment: DRI-CHEM, FUJIFILM, Model No. 3200)

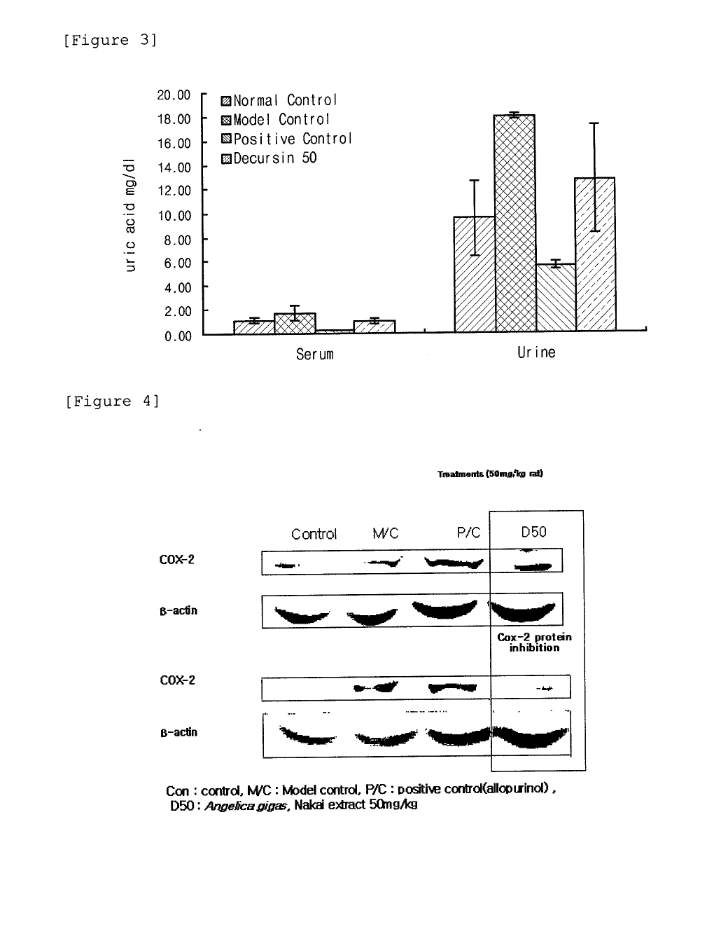

The uric acid levels in the serum and urine were found to be low, compared to the hyperuricemia control group (Model control) (see FIG. 3).

When the uric acid levels in the serum and urine were analyzed, the Angelica gigas Nakai extract-treated group (Decursin 50) showed a slightly low activity of reducing the uric acid level, compared to the allopurinol-treated group (positive control), suggesting that the effect is very beneficial, considering various side-effects of the commercially available allopurinol.

4. Test on Inflammation Inhibition in Hepatocytes of Gout-Induced Animal Model

In order to confirm the inhibitory effect of the Angelica gigas Nakai extract of the present invention on the inflammation-inducing enzyme Cox-2, the uricase inhibitor, potassium oxonate was administered to induce hyperuricemia. Then, in order to analyze the effect of reducing the blood uric acid by the Angelica gigas Nakai extract, a gout disease model was administered with an inhibitor of uric acid synthesis, allopurinol as a positive control, and used to perform a comparative experiment. β-actin was used as a control.

As the experimental animal, 9-week old male sprague-dawley rats (250-280 g, Samtako, Inc.) were acclimated in a SPF chamber where the humidity and temperature were maintained at 50±5% and 24˜26° C. and feed and water were supplied ad libitum for 1 week, and then used for the experiment.

In the present experiment, the uricase inhibitor, potassium oxonate (150 mg/kg) was intraperitoneally administered one day before sample treatment, so as to induce hyperuricemia. The contents of uric acid before and after administration of potassium oxonate were measured and compared to each other, in order to confirm the hyperuricemia induction by administration of potassium oxonate.

Potassium oxonate was suspended in 0.5% sodium carboxymethylcellulose (CMC-Na, 0.5% CMC with 0.1 M sodium acetate (pH5)), and used. For the experiment, hyperuricemia-induced experimental animals were divided into four groups, and each group consists of 7 rats: a normal control, a hyperuricemia control group (model control), an allopurinol-treated group (positive control), and an Angelica gigas Nakai extract-treated group (Decursin 50).

The hyperuricemia control group was administered with an equal amount of solvent, instead of the Angelica gigas Nakai extract. Each of allopurinol and Angelica gigas Nakai extract was suspended in PBS containing 0.1% tween 80, and orally administered for 3 days after hyperuricemia induction. The allopurinol-treated group was administered at a commercial dose of 50 mg/kg, and the Angelica gigas Nakai extract was also orally administered at a dose of 50 mg/kg.

After oral administration for 3 days, the experimental animals were fasted overnight, and then anesthetized with dry ice. A laparotomy was performed, and the livers were excised and weighed. Then, the livers were rapidly frozen and stored at −80° C. 100 mg was dissected from the whole liver, and proteins were separated using a cell lysis buffer, and the protein concentration was measured by BCA protein assay. An equal amount of protein was separated by sodium dodecyl sulfate-polyacrylamide gel electrophoresis (SDS-PAGE), and then transferred onto a polyvinylidene fluoride membrane (PVDF). After completion of the transfer, the PVDF membrane was reacted for approximately 1 hr with PBST (0.01% polyoxyethylene sorbitan monooleate-0.01M phosphate buffered saline) containing 5% non-fat dry milk, in order to block non-specific binding. Subsequently, the membrane was reacted with a primary antibody overnight, and then reacted for 1 hr with a secondary antibody against each antibody, anti-goat IgG and anti-rabbit IgG. Between each reaction, the membrane was washed with PBST for 5 min five times. After the last washing, the protein bands corresponding to each antibody were developed using a supersignal west pico chemiluminescent substrate (Pierce, Rockford, Ill., USA).

As shown in FIG. 4, it was found that Cox-2 expression in the hepatic tissue was inhibited in the Angelica gigas Nakai extract-treated group, compared to the allopurinol-treated group, and in particular, the Cox-2 expression was adversely increased in the allopurinol-treated group (positive control group), compared to the hyperuricemia control group (model control), indicating one of the side-effects of allopurinol. On the basis of the results, it can be seen that the Angelica gigas Nakai extract has an inhibitory effect on inflammation due to gout.

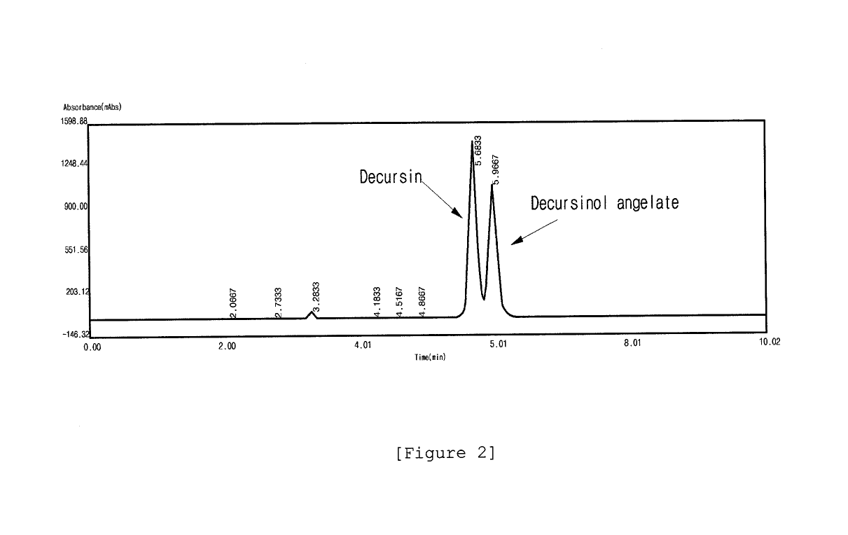

In Examples of the present invention, the Angelica gigas Nakai extract was used to perform the experiments, but an Angelica gigas Jiri extract can be also used (FIG. 2).

1. A composition for the treatment of gout, comprising an Angelica gigas Nakai extract effective in the treatment of gout by inhibition of xanthine oxidase.

2. The composition according to claim 1, wherein the Angelica gigas Nakai extract contains 98% by weight or more of decursin and decursinol angelate.

3. The composition according to claim 1, wherein the composition has 50% xanthine oxidase inhibition (IC₅₀) of 18.2 μg/ml and 90% xanthine oxidase inhibition (IC₉₀) of 44.7 μg/ml.

4. A composition for the prevention of inflammation in gout, comprising an Angelica gigas Nakai extract that has an inhibitory effect on the inflammation-inducing enzyme, Cox-2.

5. The composition according to claim 4, wherein the Angelica gigas Nakai extract contains 98% by weight or more of decursin and decursinol angelate.

6. A composition for the treatment of gout, comprising an Angelica gigas Jiri extract effective in the treatment of gout by inhibition of xanthine oxidase.

7. The composition according to claim 6, wherein the Angelica gigas Jiri extract contains 98% by weight or more of decursin and decursinol angelate.

8. A composition for the prevention of inflammation in gout, comprising an Angelica gigas Jiri extract that has an inhibitory effect on the inflammation-inducing enzyme, Cox-2.

9. The composition according to claim 8, wherein the Angelica gigas Jiri extract contains 98% by weight or more of decursin and decursinol angelate.
